(19) United States
(12) Patent Application Publication
(10) Pub. No.: US 2008/0069729 A1
(43) Pub. Date: Mar. 20, 2008

(54) Liquid Valving Using Reactive or Responsive Materials

(76) Inventor: Michael R. McNeely, Murrieta, CA (US)

Correspondence Address: MORRISS OBRYANT COMPAGNI, P.C., 734 EAST 200 SOUTH, SALT LAKE CITY, UT, 84102, US

(21) Appl. No.: 11/884,150

(22) PCT Filed: Feb. 16, 2006
(86) PCT No.: PCT/US06/05889

§ 371 Date: Aug. 9, 2007

Related U.S. Application Data
(60) Provisional application No. 60/653,566, filed on Feb. 16, 2005.
(60) Provisional application No. 60/674,476, filed on Apr. 25, 2005.

(51) Int. Cl. G01N 33/00

(52) U.S. Cl. 422/63; 422/103

(57) ABSTRACT

A technology is described for a simple, inexpensive, and easy to use sample testing platform for screening a liquid sample against a panel of different chemical or biological indicators in a robust, disposable format.

### CROSS-REFERENCE TO RELATED PATENT APPLICATIONS

This PCT application claims benefit and priority to the filing of U.S. Provisional Patent Application Ser. No. 60/653,566 filed on Feb. 16, 2005, titled “LIQUID VALVING USING REACTIVE OR RESPONSIVE MATERIALS” and also the filing of U.S. Provisional Patent Application Ser. No. 60/674,476 filed Apr. 25, 2005, titled “LIQUID VALVING USING REACTIVE OR RESPONSIVE MATERIALS” the contents of both of which are incorporated herein by reference for all purposes.

### BACKGROUND OF THE INVENTION

1. Field of the Invention

The present invention relates generally to devices for sampling fluids for storing, analysis and processing. More specifically, the present invention relates to a valve mechanism that is liquid activated, reaction chambers, reagents and structural members for containing same and related methods.

2. Description of Related Art

Accurately measuring and combining a known volume of liquid with a known volume or mass of reagents in a controlled fashion is critical in virtually all quantitative or semi-quantitative chemical or biological analyses.

Allowing for a reaction to take place and then removing excess reagents that may interfere with quantitative analysis or measurement is also important in many applications.

Being able to perform the first or both of these sample handling or reaction steps in a multiplexed fashion, in a very easy to use and inexpensive platform, would be a valuable capability and is the subject matter of this disclosure.

### SUMMARY OF THE INVENTION

The fundamental technology described in this disclosure allows for a precise volume of liquid sample to mix and react with a precise amount of reagent in a number of parallel, small volume chambers. Sample loading may be as easy as squeezing and releasing a bulb integrated within the device. When the bulb is released, the liquid sample is aspirated into the device and distributed into a number of parallel reaction chambers pre-loaded with analysis reagents. A washing step can also be integrated into the device allowing for greater sensitivity for biological analysis. Chamber filling and reactions happen automatically, making device operation easy and straightforward for possible consumer-level use.

Fluid handling or valving is performed by a controlled reaction between the liquid, stored reagents, and a valve material that will either swell, polymerize, dissolve in, or in another manner react with itself and/or with the liquid as it enters the valve, so that it will completely or partially impede the continued flow of the liquid through the valve.

The manufacturing processes used are fairly standard, allowing for low device costs in high volumes. The reagents can be chemical, biological, enzymatic, fluorescent, coated beads, compressed powders, lyophilized pellets, test strips, etc.

Result determination is performed by comparing reaction chamber fluid color with color standards within the device or on a separate chart. For some applications a spectrometer or fluorescence reader may be needed for increased sensitivity and improved quantification. A supporting instrument may also be needed for controlling more complex sample handling. Electrical based detection using embedded electronic parts within the disposable plastic testing kit is also possible.

Applications may include chemical and biological screening of drinking water, analysis of fresh or salt water aquariums for exotic fish enthusiasts or aquaculture applications, industrial waste water testing, food and beverage testing, alcohol concentration measurements, urine testing, saliva testing, screening of solvents, petrol, diesel and oil for heavy metals or other contaminants, etc.

### BRIEF DESCRIPTION OF THE FIGURES

The following drawings illustrate exemplary embodiments for carrying out the invention. Like reference numerals refer to like parts in different views or embodiments of the present invention in the drawings.

FIG. 1 shows a simple three reaction chamber testing kit according to an embodiment of the present invention.

FIGS. 2A-B illustrate a valve that utilizes the negative capillary forces exerted by hydrophobic beads and an aqueous liquid to establish a pressure barrier to prevent fluid flow according to an embodiment of the present invention.

FIGS. 3A-B illustrate a valve that utilizes the negative capillary forces exerted by hydrophilic beads and a non-polar liquid to establish a pressure barrier to prevent fluid flow according to an embodiment of the present invention.

FIGS. 4A-B show a valve where the fluid flow is stopped by a pressure barrier created by a material that absorbs the incoming liquid and swells according to an embodiment of the present invention.

FIGS. 5A-B show a valve that contains a material, typically in powder form, that will polymerize once it becomes wetted with the incoming liquid and forms a semi-solid that stops fluid flow according to an embodiment of the present invention.

FIGS. 6A-B show a valve that contains a material that dissolves in the incoming liquid to form a very viscous product that creates a pressure barrier and prevents further fluid flow according to an embodiment of the present invention.

FIGS. 7A-C show views of a temporary valve according to an embodiment of the present invention.

FIGS. 8A-C show an isolation, delayed action, or flow-through valve according to embodiments of the present invention.

FIG. 9 shows a testing kit that utilizes both temporary and permanent valves for reaction chamber washing according to embodiments of the present invention.

FIG. 10 shows a testing kit with 3 reaction chambers that are isolated from each other by flow-through valves according to an embodiment of the present invention.

FIG. 11 shows a device with a positive and negative color control chamber and chambers for six different potential titration concentrations of a particular analyte.

FIG. 12 shows a design of a simple single reaction chamber device according to an embodiment of the present invention.

FIG. 13 shows a testing kit with an integrated particle or bubble filter and an integrated leur-lock fitting for syringe attachment according to an embodiment of the present invention.

FIG. 14 shows a testing kit with a syringe connection where the syringe is used to aspirate the sample into the testing kit instead of an integrated bulb according to an embodiment of the present invention.

FIG. 15 shows a device with an integrated needle for collecting a sample through a membrane according to an embodiment of the present invention.

FIG. 16 shows a device for collecting three samples simultaneously with a single bulb aspiration according to an embodiment of the present invention.

FIG. 17 shows a device with an integrated pipette connection point for collecting three samples simultaneously according to an embodiment of the present invention.

FIG. 18 shows a three analyte testing kit with syringe loading and capable of washing according to an embodiment of the present invention.

FIG. 19 shows a three analyte testing kit with two inlets for changing the inlet liquid source according to an embodiment of the present invention.

FIG. 20 shows a testing kit capable of bulb aspiration for sample loading as well as a syringe attachment, such as for wash buffer delivery according to an embodiment of the present invention.

FIG. 21 shows a testing kit with an integrated sample collection cup according to an embodiment of the present invention.

### DETAILED DESCRIPTION OF THE INVENTION

Embodiments of the present invention are not meant to be limited by the nature of the liquid sample or the nature of the chemical or biological reactions that are to take place, or the method of detecting the reaction products. This disclosure is not meant to be limited by the sample volumes, testing kit or channel or reaction chamber dimensions, or materials from which the testing kit is made or the materials used to perform the valving methods described.

One aspect of the present invention is to permanently or temporarily stop or slow the flow of a liquid traveling in an enclosed channel by having it pass through a valve that is comprised of a material that will either swell, polymerize, dissolve in, or in another manner react with itself or with the liquid as it enters the valve, either passively or actively, so that it will completely or partially impede the continued flow of the liquid through the valve.

Other aspects of the present invention include the liquid chemical and physical properties, the valve dimensions, the mechanism that causes fluid flow, and the valve material chemical and physical properties. Pressure driven flow, such as by positive pressure or vacuum driven sources, is the focus of this discussion, but other flow mechanisms may also be used.

A further aspect of the present invention is to have a liquid sample be accurately aliquoted into multiple smaller samples that then enter and fill reaction chambers where reagents that react with the incoming liquid have been stored. In this manner, a panel of analyses can be performed on a liquid sample in a one-step process. An “aliquot” is a measured portion of a sample taken for analysis.

A precise volume of liquid may be aliquoted by accurately controlling the volume of the reaction chamber that is formed in the testing kit. The liquid fills the reaction chamber and, in most applications, does not flow out of the reaction chamber.

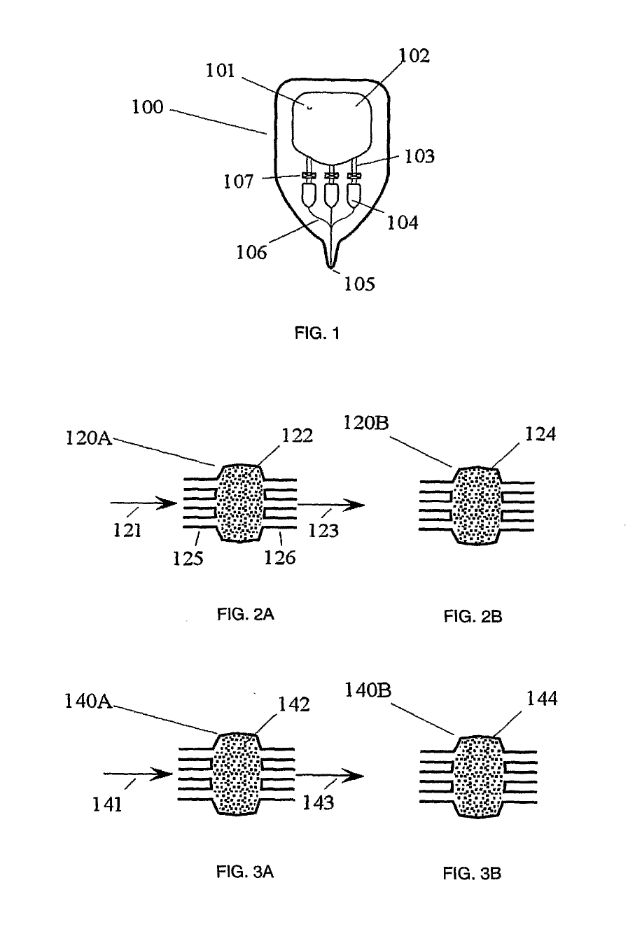

FIG. 1 shows a simple three reaction chamber testing kit 100, according to an embodiment of the present invention. A thumb aspiration bulb 102 is integrated into the device with a pressure release hole 101 that is covered during aspiration. When the bulb is pressed, air is pushed out the air channels 103 and exits the device. The inlet 105 is placed in the liquid sample and the thumb bulb is slowly released. Liquid fills the device through the liquid channels 106 and fills the reaction chambers 104. Once aspiration is completed and all reaction chambers are filled, the pressure release hole is uncovered to release any remaining suction pressure in the bulb. The valves 107 ensure each of the reaction chambers is filled properly and prevent any liquid flow into the bulb.

FIGS. 2A-B are diagrams of a valve 120A and 120B, respectively, that utilizes the negative capillary forces exerted by hydrophobic beads 122 and an aqueous liquid to establish a pressure barrier to prevent fluid flow, according to another embodiment of the present invention. FIG. 2A shows the valve 120A with hydrophobic beads 122 and the direction of liquid flow 121 as air 123 is drawn out through the valve 120A. There are redundant inlets 125 and redundant outlets 126 to minimize the occurrence of flow stoppage due to clogged channels and reduce the likelihood of valve material leaking from the valve area. FIG. 2B illustrates flow stoppage in valve 120B due to the pressure barriers created by the negative capillary forces 124 between the aqueous liquid and hydrophobic beads 122.

FIGS. 3A-B are diagrams of a valve 140A and 140B, respectively, that utilizes the negative capillary forces exerted by hydrophilic beads 142 and a non-polar liquid to establish a pressure barrier to prevent fluid flow, according to another embodiment of the present invention. In FIG. 3A, air 143 drawn into the released bulb (not shown) aspirates a non-polar liquid 141 into the valve 140A. In FIG. 3B, due to the pressure barriers created by the negative capillary forces 144 between the non-polar liquid 141 and hydrophilic beads 142, fluid flow stops within valve 140B.

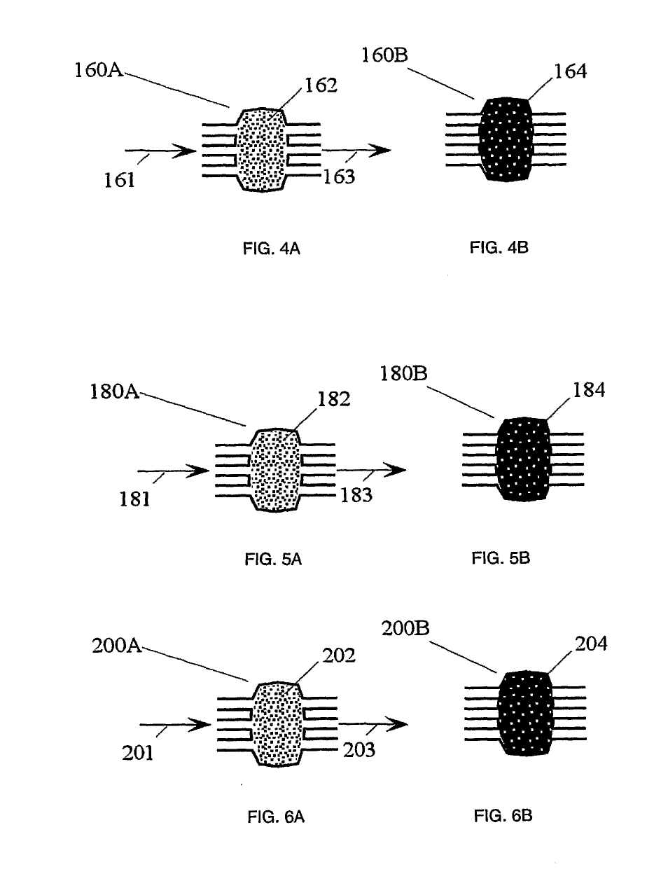

FIGS. 4A-B shows a valve 160A and 160B, respectively, where the fluid flow 161, drawn in by the aspiration force 163, is stopped by a pressure barrier created by a valve material 162 that absorbs the incoming liquid and swells 164 (FIG. 4B, darkened region), according to another embodiment of the present invention. One example of a valve material 162 is hydrogel powder that can absorb a considerable amount of water (fluid flow 161) and swells 164 to fill the space within the valve well, thus blocking fluid flow 161 through the valve 160B. Of course, other suitable valve materials 162 may also be known to those skilled in the art and are considered to be within the scope of the present invention.

FIGS. 5A-B illustrate diagrams of a valve 180A and 180B, respectively, that contains a material 182, typically in powder form, that will polymerize 184 once it becomes wetted with the incoming liquid 181, which is drawn in by the aspiration force 183, according to another embodiment of the present invention. The material may contain a polymerizable material, as well as catalysts that enhance the rate of polymerization. The materials must be soluble in the incoming liquid, or in a product of the incoming liquid and a reactant product of a component of the valve material and incoming liquid. One example of material 182 is “EnviroBond™ 403” that polymerizes with itself in certain non-polar solutions and solvents. However, this embodiment of the present invention is not limited to material 182 comprised of EnviroBond™ 403, as any suitable polymerizing material 182 may be used consistent with the present invention.

FIGS. 6A-B illustrate diagrams of a valve 200A and 200B, respectively, that contains a material 202 that dissolves in the incoming liquid 201 to form a very viscous product 204, according to another embodiment of the present invention. The viscosity of the resultant product serves as a pressure barrier to continued flow of the liquid through the valve 200B and the valve's small exit channels (such as 126 on FIG. 2A). An example of a suitable material 202 for this valve type (200A and 200B) is high-viscosity carboxy methyl cellulose powder.

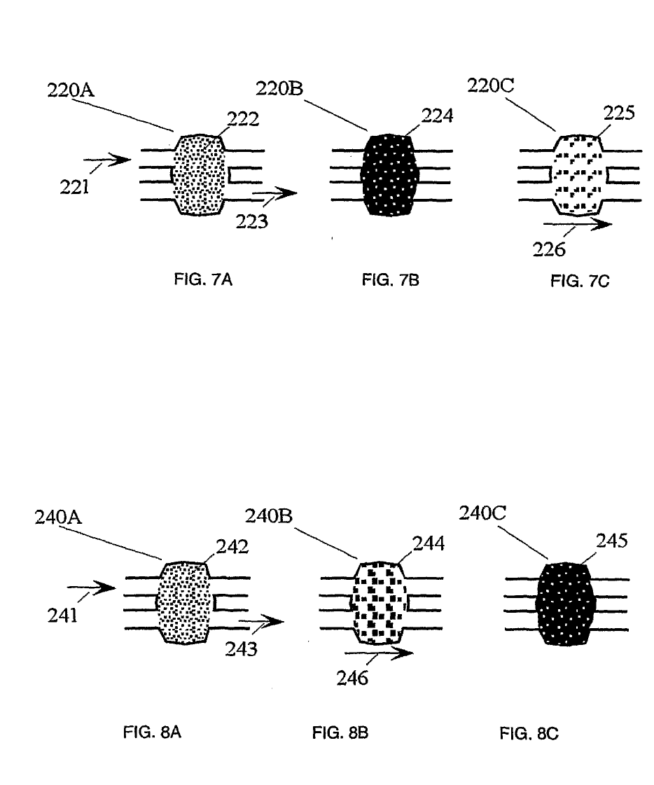

FIGS. 7A-C illustrate diagrams of a valve 220A-C, respectively, that is non-permanent, according to another embodiment of the present invention. In valve 220A, aspiration force 223 draws liquid 221 into the valve area containing valve material 222. In valve 220B the valve material 222 reacts with or responds to the incoming liquid 221, such as by swelling, to form a pressure barrier 224 against fluid flow. After a predetermined amount of time, the valve material 222 in valve 220C then degrades 225, e.g., by dissolving or reacting with a slower acting secondary valve material, to release its pressure barrier (224 in FIG. 7B) and allow flow 226 to resume as shown in FIG. 7C. An example of a suitable valve material 222 is starch that may absorb the incoming liquid 221, and then swell to prevent fluid flow. After time, the swollen starch may dissolve in the liquid, or a starch digestive enzyme or amylase may be present that actively breaks down the swollen starch, which releases the pressure barrier 224.

FIGS. 8A-C illustrate diagrams of a valve 240A-C, according to another embodiment of the present invention that reacts with the incoming liquid 241 in any of the ways described in FIGS. 2-6, but the described reaction is delayed for a specific duration before a sufficient pressure barrier is established to prevent fluid flow. The specific duration may be in the amount of time required for the material 242 to respond sufficiently, or in the volume of liquid 241 required to pass through the valve 220A before it stops further liquid flow. In valve 240B the liquid flow slows 246 due to the reacting valve material 244. In valve 240C the material 245 is fully reacted, the flow stops and the aspiration force 243 has no further effect.

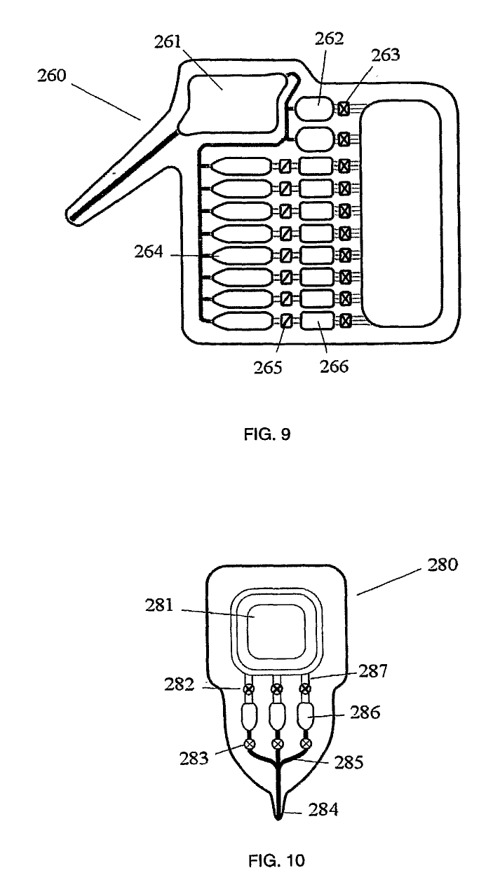

FIG. 9 shows a device 260, according to another embodiment of the present invention that includes a wash buffer well 261 that stores some of the incoming liquid for use as a washing or rinsing solution once the liquid is aliquoted out and fills all of the reaction chambers 264. The temporary valves 265 serve to distribute the liquid across all of the reaction chambers 264. As the temporary valves 265 give way, fluid fills the dead-volume wells 266 and is prevented from further advancement by the permanent valves 263. The volume of the dead-volume wells 266 is the volume of liquid that will wash through the reaction chambers 264. Aliquots of the unwashed liquid are kept in wells 262.

FIG. 10 shows a testing kit 280, according to another embodiment of the present invention with three reaction chambers 286 that are isolated from each other by flow-through valves 283. When the thumb bulb 281 is depressed, air exits the testing kit 280. The tip 284 (or inlet 284) of the testing kit 280 is placed in the liquid to be loaded. As the thumb bulb 281 is released, the liquid is drawn into the testing kit 280 through the liquid channels 285 and through the flow-through valves 283. The liquid fills the reaction chambers 286 and is distributed evenly across them and prevented from filling the air ducts 287 and bulb region 281 by the permanent valves 282. The isolation valves 283 then seal and physically and chemically isolate each reaction chamber 286 from each other.

FIG. 11 shows a titration test kit 300, according to another embodiment of the present invention with a positive 304 and negative 305 color control chamber and reaction chambers 308 for six different potential titration concentrations of a particular analyte. The bulb 301 is depressed, forcing air out of the device. The tip 306 is placed into the liquid and the bulb 301 is released. Liquid fills the kit through flow channels 307 and is distributed across all reaction chambers 308 by the permanent valves 302. The permanent valves 302 also prevent the liquid from flowing through the air ducts 303 into the bulb 301. In this manner a precise volume of liquid will fill each reaction chambers 308 and react with reagents that may be located within each reaction chamber 308.

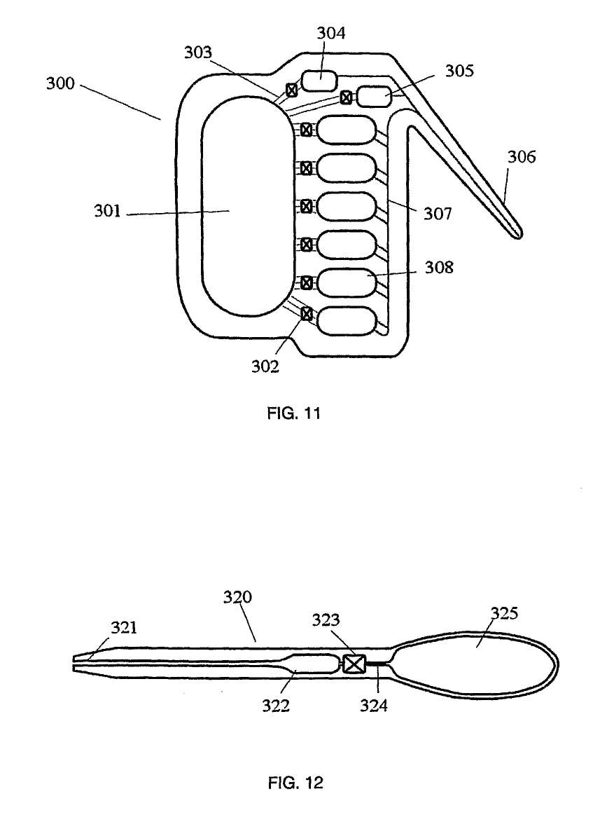

FIG. 12 shows a design of a simple single reaction chamber device 320, according to another embodiment of the present invention. The bulb 325 is pressed, forcing air out of the single reaction chamber device 320. As the device inlet 321 is placed into the liquid, the bulb 325 is released and liquid fills the reaction chamber 322. The liquid is contained within the reaction chamber 322 and prevented from entering the air duct 324 leading to the bulb 325 by the action of the permanent valve 323.

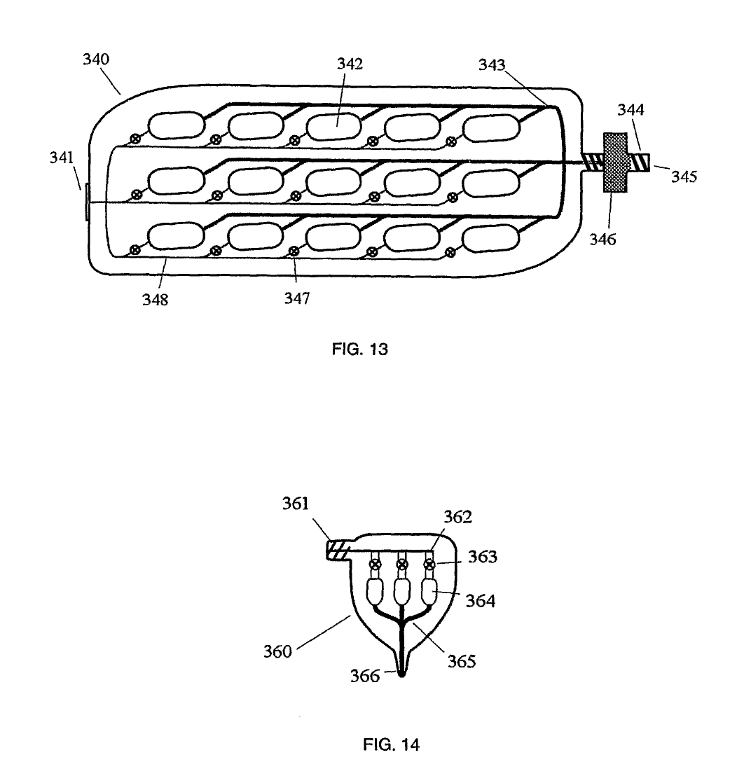

FIG. 13 shows a syringe-loaded, multiple analyte testing kit 340, according to another embodiment of the present invention. A syringe (not shown) is connected to the testing kit 340 at the leur lock fitting 344 at the device inlet 345. Liquid within the syringe is dispensed through a filter 346 into the liquid channels 343 that lead to the multiple reaction chambers 342. The permanent valves 347 ensure the liquid is evenly distributed and fills all reaction chambers. As the testing kit 340 fills with liquid, air is displaced out of the testing kit 340 through the air channels 348 and through the air vent 341. The air vent 341 may have a membrane covering it to prevent particles from entering and blocking the air channels 348.

FIG. 14 shows a testing kit 360, according to another embodiment of the present invention with a syringe connection 361, where the syringe (not shown) is used to aspirate the liquid sample into the testing kit 360 according to an embodiment of the present invention rather than an integrated aspiration bulb as described in a previous embodiment. The inlet 366 is placed in the liquid and the syringe plunger (also not shown) is drawn out, aspirating the liquid through the flow channels 365 and into the reaction chambers 364. The permanent valves 363 prevent liquid from flowing out of the reaction chambers 364 and into the main air duct 362 and the syringe.

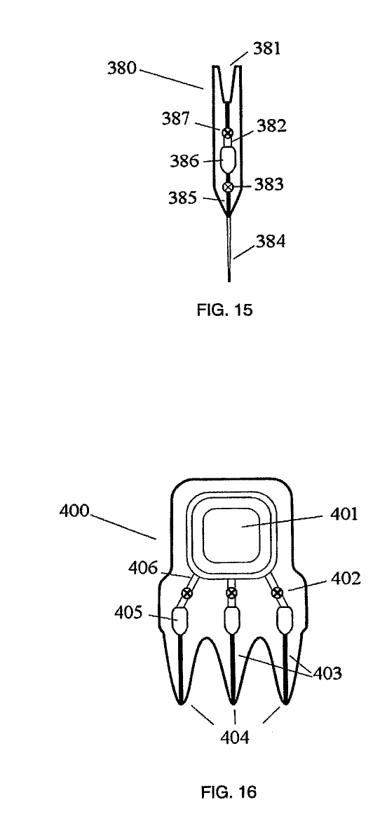

FIG. 15 shows a single sample device 380 that has a fitting for pipette aspiration 381 and a needle 384 for puncturing a membrane or vein (not shown) to gain access to a liquid, according to another embodiment of the present invention. Single sample device 380 also has an isolation valve 383 for sealing the liquid sample in the sample chamber 386. Liquid is drawn through the flow channels 385 into the sample chamber 386. Small channels 382 minimize the volume of liquid that leaks into the permanent valve 387 before it seals.

FIG. 16 shows a three sample kit 400, according to another embodiment of the present invention. An aspiration bulb 401 is connected through air ducts 406 and permanent valves 402 to single reaction chambers 405 for each liquid sample. The tips 404 of the sample kit 400 are placed in the samples and the liquid is drawn up through flow channels 403 into the sample kit 400.

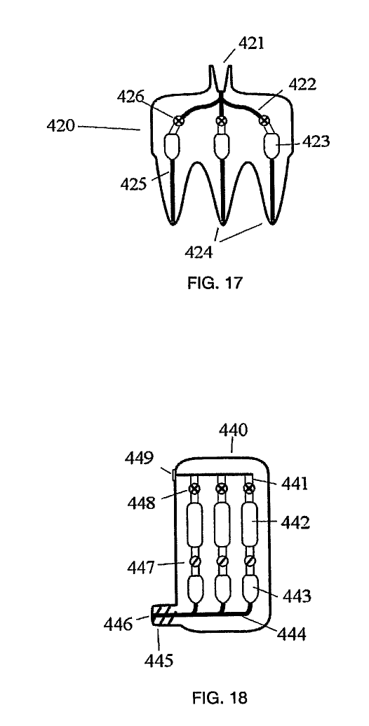

FIG. 17 shows a multi-sample kit 420 with a pipette fitting 421 for aspirating the samples into the multi-sample kit 420, according to another embodiment of the present invention. Permanent valves 426 prevent the liquid from entering the air channels 422. The permanent valves 426 also ensure the aspiration force continues on each inlet 424 until its corresponding reaction chamber 423 is filled.

FIG. 18 shows a three chamber testing kit 440 with a leur lock fitting 445 for syringe attachment at the device inlet 446, according to another embodiment of the present invention. Liquid is dispensed through the liquid channels 444 into the reaction chambers 443. The temporary valves 447 evenly distribute the liquid into all reaction chambers 443. At this point, if desired, the syringe (not shown) can be swapped out for another syringe (also not shown) that contains a wash buffer. The liquid from the original or new syringe is then dispensed into the three chamber testing kit 440, pushing the original liquid sample through the temporary valves 447 and into the dead-volume wells 442. The volume of the dead-volume wells 442 defines how much the reaction chambers 443 are rinsed with further liquid. As liquid enters the three chamber testing kit 440, air is vented through the permanent valves 448 and through the air ducts 441 and out of the three chamber testing kit 440 at the air vent hole 449.

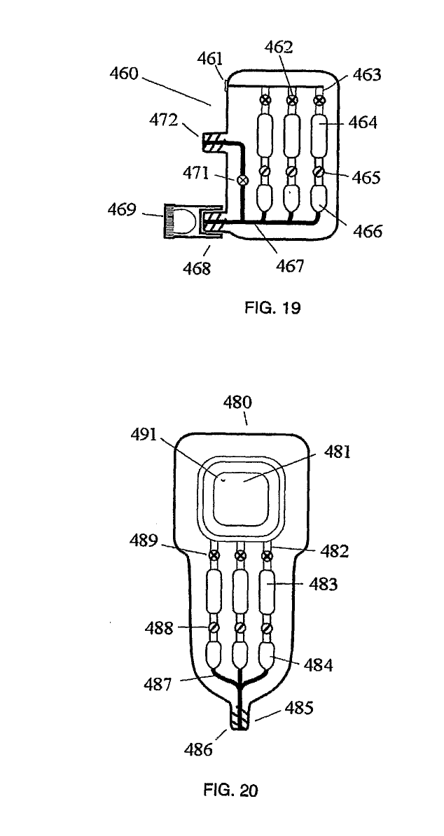

FIG. 19 shows a two inlet device 460, according to another embodiment of the present invention. A syringe may be connected at the first inlet 472 and liquid is dispensed into the device through the flow channels 467. Temporary valves 465 allow the first liquid to be distributed evenly into the reaction chambers 466. Liquid flowing through the first inlet 472 may also leak out the second inlet 468, but is prevented from significant leaking due to the bleed-through cap 469. The cap 469 can be removed and a second syringe attached that dispenses a secondary reagent or wash solution. The secondary solution is prevented from leaking out the first inlet 472 due to an isolation valve 471 that blocks that exit. Liquid is pushed past the temporary valves 465 and into the dead-volume wells 464 where it is prevented from entering the air ducts 463 by the permanent valves 462. Air escapes the device at the air vent 461.

FIG. 20 shows a dual bulb and syringe device 480, according to another embodiment of the present invention. Liquid is loaded into the device 480 by covering the pressure release hole 491, pressing the bulb 481, putting the inlet 486 of the device 480 into the liquid and then releasing the bulb 481 without uncovering the pressure release hole 491. Once the liquid fills the reaction chambers 484 and is distributed and stopped by the temporary valves 488, the pressure release hole 491 can be uncovered. At this point a syringe (not shown) can be connected at the leur lock fitting 485 at the device inlet 486. As a secondary liquid is dispensed into the device 480, the remaining air in the device 480 is vented through the air ducts 482 and through the pressure release hole 491. The secondary liquid pushes the liquid sample past the temporary valves 488 until all dead-volume wells 483 are reached and the permanent valves 489 seal.

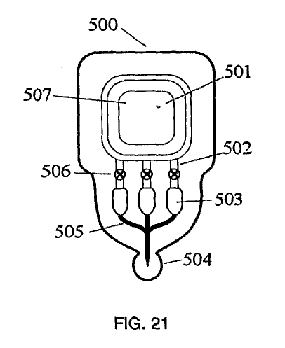

FIG. 21 shows a three reaction chamber device 500 with an integrated bulb 507 and an integrated sample collection cup 504 at the device inlet, according to another embodiment of the present invention. Pressure release hole 501 may or may not be necessary. The sample collection cup 504 is filled with the liquid sample. Flow channel 505 draws the sample from the base of the cup 504 and fills the reaction chambers 503. Permanent valves 506 prevent liquid from entering the air ducts 502 and filling the bulb 507.

Referring again to FIG. 1, a bulb section 102 of an injection molded plastic testing kit 100 may be connected through air channels 103 (or air ducts 103) to wells designed to act as valves 107. As the bulb 102 is compressed, e.g., with the fingers, air is displaced out of the bulb 102, through the air ducts 103, valves 107, reaction chambers 104 and fluid channels 106 into the atmosphere. The tip 105 (or inlet 105) of the three reaction chamber testing kit 100 is then placed into a liquid source (not shown) and the finger pressure is slowly released. The liquid may then drawn into the fluid channels 106 and into the reaction chambers 104. Each reaction chamber 104 is filled and eventually the liquid enters the air duct 103 leading to the valves 107 where the liquid interacts with the material in the valve wells 107. The valve wells 107 are designed to allow air to pass through when the material is dry, but once wetted by the liquid, the valves 107 react or respond to prevent further air and liquid passage.

As one reaction chamber 104 is filled and its corresponding valve 107 closes, the remaining vacuum suction of the bulb 102 will draw the fluid through the remaining channels 106 and cause the other wells 107 to fill in the same manner.

When all of the reaction chambers 104 are filled there still may be suction force in the bulb 102. This suction force can be vented either by uncovering a small hole 101 in the bulb 102 that was previously covered by the fingers, or by puncturing the bulb to vent the vacuum force, or the valves 107 must be able to contain this suction pressure either indefinitely, or for a time that is sufficient for whatever reaction is needed to come to completion, the results to be read and the testing kit disposed of properly.

In FIG. 1 three reaction chambers 104 are present, such as for a positive and negative control, and the unknown sample. FIG. 1 also includes a vacuum release, or pressure release, or vacuum venting hole 101. Normally this would be covered with a finger as the bulb is compressed and the bulb 102 is slowly released. Once all reaction chambers 104 are filled the pressure release hole 101 can be uncovered completely to vent the remaining vacuum pressure that may be present.

Valve Mechanisms and Materials: Depending on the liquid type and valving requirements, there are many different valving materials and mechanisms that can be considered. In each case, and referring to FIGS. 2A-B as an example, the valve 120A consists of a small well with inlet 125 and outlet 126 channels or air ducts. The air ducts and valve wells are as small as possible and as small as practicable to minimize the dead volume of the valve and to reduce the chance of material leaking out of the valve well. The valve is filled with pellets, micro or nano beads, powder, or a similar form of material 122. There is often a redundancy in the inlet and outlet channels to ensure at least one channel remains patent and is not blocked by the valve material or another substance. There may also be a redundancy in the valves to ensure that the flow out of one reaction chamber is stopped. This may have the detrimental effect of increasing the dead volume of the valve, but it may be necessary in some cases. This may also be needed if the liquid contains many complex components, not all of which would respond to just one valving mechanism.

For some applications there may be redundancy in the valves downstream of the reaction chamber and the first valve may only be needed to temporarily slow or stop flow to properly aliquot the sample. In this case the total dead-volume of the valve may be designed to be large so as to create a reaction chamber washing effect. The first valve allows for accurate fluid distribution and aliquoting, whereas the second valve controls the washing step volume.

FIGS. 2A-B shows a type of valve that uses negative capillary forces to generate a pressure barrier to fluid flow. In order for negative capillary forces to exist, the liquid needs to be aqueous with minimal surfactants, solvents, or aliphatic components, and the valving material needs to be strongly hydrophobic (HFB). Suitable materials can be Teflon™ powder or micro or nano beads with hydrophobic coatings. The porosity of the valve material plug needs to be very small, sub-10 μm and preferably sub-1 μm, to allow for a strong enough pressure barrier to stop flow generated by bulb suction.

In this case, aliphatic is meant to refer to compounds that may contain both polar and non-polar components, making them soluble or partially soluble in both polar and non-polar liquids and, generally, non-responsive to capillary forces.

It is conceivable that a hydrophobic membrane can also be inserted and sealed across the flow path to act as a HFB valve. Also, it is possible that the base material of the cassette, or at least the valve region, can be made of a hydrophobic plastic, or coated with a hydrophobic film, with the valve consisting of just a small channel. The channel dimensions in this case will need to be extremely small, sub-1 μm, in order to withstand the potential bulb vacuum pressure. Although possible, neither the HFB membrane nor the HFB base plastic or film methods necessarily lend themselves to be economically manufacturable.

If non-polar solutions are used, such as oils, the valve material needs to be strongly hydrophilic (HFL). FIGS. 3A-B show a hydrophilic valving type. The same valve material plug porosity as described in the preceeding paragraphs needs to be present in this case as well. Suitable strongly hydrophilic materials include glass beads. The non-polar solution should not contain polar solvents in any great concentration, nor should aliphatic compounds be present in any great quantity.

The pressure drop that needs to be withstood by the valves in the case of bulb aspiration may reach, but will not be greater than, atmospheric pressure, or 14.7 psi. It may be substantially less than this, depending on bulb design, material, and air and liquid flow rates.

FIGS. 4A-B show a valving type where the valve material 162 is designed to swell in the presence of the incoming liquid and block advancement of the liquid through the air ducts. Examples of appropriate liquid material combinations include water and acrylamide hydrogel (HGL) powder.

In most instances the valve material needs to be either in powder or small granule form to maximize the surface area of liquid interaction and to maximize the speed of valve response. In the HFB and HFL cases, the materials need to be small to minimize porosity, so as to maximize the pressure barrier. Large granule hydrogel pellets or hydrogel films will not respond quickly enough to stop flow in any reasonable time scale. By the time they may respond, the incoming liquid will likely have washed out and diluted the reagents needed for an accurate chemical reaction.

There are many types of hydrogel materials that respond to different aqueous solutions. Some HGLs may be customized for acidic solutions (Enviro-Bond™ 300-22A), basic solutions (Enviro-Bond™ 300-22C), high salt content solutions (Goodfellow™ AC336310 Polyacrylamide/acrylate hydrogel), and some are functionalized to respond to more complex environments.

In addition there are many plastics that swell in the presence of solvents (PVC), which may be suitable material combinations for the processing of liquids that contain a high concentration of solvents, such as gasoline.

Another valve mechanism shown in FIGS. 5A-B is one where the valve material 182 reacts with itself and/or the incoming liquid when triggered by something in the incoming liquid 181 or a reagent that is stored in dry format that dissolves in the incoming liquid 181. EnviroBond™ 403 is an example of a material that polymerizes in the presence of certain organic liquids such as crude oil, diesel fuel and gasoline. EnviroBond™ 403 powder polymerizes with and encapsulates these organic liquids and forms a semi-solid sponge-like material that can impede or block fluid flow.

A quick responding two part adhesive may respond in a similar manner. One part could be in the reaction chamber and the other part exist in powder form in the valve well. Both parts would likely need to be soluble in the liquid to be analyzed.

Another valve mechanism shown in FIGS. 6A-B is one where the incoming liquid 201 dissolves the valve material 202, but the resultant solution 204 is highly viscous and blocks the further flow of liquid. An example of this is high viscosity carboxymethylcellulose sodium salt (CMC) (Fluka BioChemika™ 21903). The viscosity of this material is so great that it immediately stops further flow and prevents further dilution of itself. Much of the viscosity is due to the material crosslinking with itself, but in this case the material remains soluble rather than polymerizing as in FIG. 5B.

Another valve material and mechanism shown in FIGS. 7A-C is a temporary valve 220A that may initially swell and or dissolve into a viscous solution to stop or slow flow for a short time (see FIG. 7B, valve 220B), but will eventually dissolve further and reduce its ability to support a pressure differential and the material and liquid will flow downstream of the valve well 220C. Such candidates include lower viscosity CMC salts (Fluka BioChemika™ 21900), starch (Sigma™ S2630) and some sugars such as Ficoll™.

In the case where a pressure release valve is present in the bulb structure, the temporary valves may function practically as permanent valves as well.

Temporary valves may also include chemicals that ultimately break down the valve material by an enzymatic process, rather than just allow the valve material to dissolve passively. Such materials include various cellulases, amylases, or xylanases. Enzymatic degradation of a valve material will require optimization of the pH and temperature of the reacted or responded valve constituents, but various reactive salts for adjusting the pH and temperature can be included in the chemicals comprising the valve material.

It is possible that non-responsive valve materials may be used, such as micro or nano chromatography beads, simply as a means of reducing the flow cross section and hence slowing flow. In some applications this may be acceptable, but it has the detrimental effect of not stopping flow entirely, but just slowing it, which may cause any further steps, such as washing, to take place at a particularly slow rate. However, it may also be useful as a type of timing mechanism.

A final valve type can be described as an isolation or pass-through valve. This is shown in FIGS. 8A-C. According to this embodiment, the valve material 242 may be either the swellable, polymerizable, or soluble valve types described earlier, but in this case the valve material is slowly responsive or reactive to the liquid sample and is placed upstream of the reaction chamber. According to this embodiment, the liquid sample may flow past the isolation or pass-through valve to a downstream stopping point 240B, and then the isolation or pass-through valve closes 240C. The delay in closure is due to the slow responding nature of the valve material 244. This will allow the sample that has already passed through the valve to be isolated from the sample liquid upstream of the valve. Isolation may be important in cases of quickly diffusing reagents, when thermal processes may take place, such as in enzyme assisted amplification, or to reduce the likelihood of stored reagents leaking out of the reaction chamber.

FIG. 9 is an example of a fluid processing circuit that uses temporary valves 265 and permanent valves 263 to distribute the liquid among eight reaction chambers 264 and two calibration chambers 262. The temporary valves 265 can allow sample incubation in the reaction chamber 264 from a few seconds to a few minutes before the temporary valves 265 dissolve or are washed away and flow resumes to fill the dead-volume wells 266. This is done to wash away excess or unreacted reagents from the reaction chamber 264. This may be done to terminate a reaction or to remove background signals that may interfere with reaction product detection. This is common in many enzymatic or immunodiagnostic based reactions where fluorescently labeled reagents may be present. FIG. 10 shows a testing kit with 3 reaction chambers 286 that are isolated from each other by flow-through valves 283.

An isolation valve may also function as a flow re-direction valve. In the case where there may be two inlets into a reaction system, the isolation valve will isolate one inlet from the other. This will allow flow to be shifted from one inlet to the other when bulb suction is used. When syringe pressure is used (described later), the isolation valve will prevent fluid from leaking out of the cassette via the initial input.

Reagents and Reaction Types: Within the reaction chamber a known mass of a single reagent, or a mixture of multiple reagents can be pre-stored. The reagents can be chemical, biological, enzymatic, fluorescent, coated beads, compressed powders, lyophilized pellets, test strips, etc. They are designed to be stable and to not react with each other in their stored form. They should be readily dissolvable in the liquid to be analyzed, or dissolvable in a reaction product of the liquid and one or more of the reagents. Many reagents that may react with each other in liquid form are perfectly stable and non-reactive in dry form. It is possible that they could be encapsulated as well and be in fluid form, where the capsule will dissolve in the liquid, or in a reaction product of the liquid and reagents.

These reagents may be either stored together under vacuum, or with protective coatings, or in separate chambers leading into one another. Likely the testing kit will be vacuum packed or sealed to improve the shelf life of the reagents and prevent them from becoming wet and reactive. Reagents can be loaded using fairly standard and straightforward assembly technology.

Chemicals that may catalyze the valve material to respond quickly may also be stored in the reaction chamber. For example, a powder basic salt such as sodium hydroxide and an acidic salt such as hydrogen sulfate may be stored together or in close proximity and will not react with each other if stored in a dry format. However, once dissolved, they can react to form neutral salts and water, but the reaction is exothermic. An increase in liquid temperature could cause the valve material to react more quickly to stop the exit of liquid from the reaction chamber.

Given the small volumes of most applications (5-250 μL) reagent mixing with the sample liquid happens fairly efficiently by diffusion. In some cases, such as with larger volumes, the reaction chamber can be designed such that a bubble remains when the chamber is filled. The cassette can then be shaken, or inverted, and the bubble can facilitate mixing. Mixing particles, such as magnetic beads, can also be stored in the reaction chamber if needed.

The reagents may differ from reaction chamber to reaction chamber to analyze different parameters of the sample liquid. Some reactions may be for the detection of biological targets while others for chemical targets. Some reactions may be for calibration of the incoming liquid, for screening for interferents of other reactions in the same kit, or for positive or negative controls.

Another set of reactions that can be performed easily by this testing kit platform are titration reactions. Each well can have a different mass of reagents designed to titrate different concentrations of a target analyte in the sample liquid. The well that has the gross color change would be identified as the approximate concentration of the analyte in the sample liquid.

FIG. 11 shows a device, according to the present invention with a positive 304 and negative 305 color control chamber and chambers 308 for six different potential titration concentrations of a particular analyte. FIG. 12 shows an embodiment of a simple single reaction chamber device 320, according to the present invention.

Detection Methodologies and Instrument Support: Many detection methodologies can be employed for measuring reaction products. The simplest is visual calorimetric, where an individual is able to identify gross color changes for titration reactions or gradual color changes for other reactions, usually with the aid of a chart identifying the analyte concentration associated with a particular color or shade.

Beyond visual calorimetric measurements a wide range of options exist, which will usually require the aid of an external instrument. These include optical based methods such as fluorescence, chemiluminescence, optical absorption, optical scattering, and spectroscopy. They may also include electrical or magnetic based measurements such as conductivity, resistivity, Hall Effect, electric potential, etc.

Ion sensitive electrodes could possibly be used, but not only where the electrodes detect an ion in the sample liquid, but potentially an ion produced by the reaction of the sample liquid with reagents stored in the reaction chamber of the testing kit.

For electrical measurements an instrument may need to interface directly with the kit, whereas optical measurements could be indirect, but require that the kit be made out of suitable optical materials.

The flexibility of this technology allows the kit to be made out of a wide range of potential materials, with a wide range of additional features and interfaces for instrumental, measurement, and fluid control. For example, the reaction chambers can be made out of the same material as optical cuvettes, and be shaped in an appropriate manner for accurate optical measurements. Conductive elements can be molded into the testing kit, such as by insert molding, for interfacing to an instrument for electrical detection or to control more advanced fluidic processing such as electrokinetic sample manipulation. Remote valving technology for advanced fluid handling may also be used, where the testing kit interfaces to external valves and pumps to control fluid movement.

Design, Manufacturing and Assembly: Generally, the testing kit will be manufactured by injection molding of a suitable plastic material. It may consist of two parts, a top and a bottom. In some cases one plastic is used for both sides, or different materials are used. For example the bottom may be made of a plastic that has well defined characteristics for geometrical accuracy, such as may be needed for the smaller air ducts (PMMA). It may also be made of a suitable material to provide chemical inertness to the sample liquid or reagents (PP), or be capable of bonding or adsorbing biomolecules to the surface of the reaction chamber (PS). It may also be colored for better optical detection or aesthetic reasons. The top may be made of a suitable material or materials to allow for transparency over the reaction chambers (PC), and flexibility over the bulb region (LDPE). Two shot molding may be used for the top or bottom to provide optimal characteristics in different parts of the cassette. Insert molding may be used for embedding an electrical element into the plastic.

A presently preferred embodiment of this device is one that can be manufactured and assembled as simply and inexpensively as possible. It may be difficult to insert the valve materials into the valve well if they exist in powder form. If the valve material is soluble in aqueous liquid, but not organic solvents, then it is possible to make a slurry of the material in an organic solvent to assist in the loading of the valve well with the valve material. As the solvent evaporates, the powder is contained in the wells without having been scattered across the whole device. Similarly, an aqueous solvent may be used for non-polar reagents.

It may be helpful to fabricate a ridge around the valve well to assist in containing the valve material. The air ducts could cut through the ridge and the ridge and well could be sealed above by adhesive and the top portion of the device.

The valve well may be round or square, shallow or deep, whatever shape is needed to facilitate its function and allow for economical manufacture and, possibly, automatic loading of the valve material.

Care may need to be taken to eliminate static charges that can build up on the plastic or adhesive surfaces. Static can cause the powder material to jump out of the valve wells and scatter across the surface of the part.

The valve material may only be available in granule or pellet form, instead of powder. To increase the speed of reaction, the material may need to be ground and filtered to an optimal size for use as a valve. This may be done in a commercial grinder or manually by a mortar and pestle. Thermal effects of grinding may adversely affect the material and care should be taken in this process.

Depending on the dimensional detail and design of the device, the top and bottom portions can be sealed together using ultrasonic welding, solvent bonding, or by lamination using transfer film, double sided tapes, or screen printed adhesive (Three Bond™ 1549 Screen Printable PSA).

The dimension of the testing kit can be optimized for specific applications, such as portability, specific sample size, optimized flow rates, etc. The bulb region should be around 3 times the volume of the total chamber and channel volume to ensure filling the chambers effectively.

The volume of the reaction chambers may range from a couple microliters to a few milliliters. This will depend on the concentration of the analyte of interest, desired accuracy, detection methods, desired sensitivity, total number of reactions per device, etc.

The dead-volume of the valve should be as small as possible to increase the accuracy of the device. A valve well 1 mm×2.5 mm×2 mm has a volume of 5 μL, but it is mostly full of the valve material powder. Given these dimensions the valve well and air duct volume may be less than 1 μL or up to about 2 μL. If the reaction chamber volume is 100 μL, and the valve dead-volume is around 1 μL, the dead-volume may account for up to about 1% of error in the reaction, which is quite acceptable for most applications. However, given the potential error associated with manufacturing variabilities, plastic shrinkage, assembly variations, as well as the variations in reagent dosage accuracy and consistency, the total potential accuracy of the device would certainly be greater than 1%. Care must be taken that applications are developed that will find the potential measurement error acceptable.

Syringe Loading: Bulb aspiration has been described as the liquid driving force. Other options also exist. For example, a sample may be aspirated into a syringe that is then connected to the inlet of the testing kit.

FIG. 13 illustrates a testing kit 340 with a leur lock syringe connection 344. The syringe may be connected to a particle or bubble filter 346 to eliminate unwanted components from the test liquid. Bubbles are often present in saliva samples, or samples collected with a sponge. A size exclusion filter may be needed for environmental samples that may contain unwanted macro particles.

A syringe loading method may be needed if the sample cannot be easily loaded into the testing kit via the bulb aspiration method. For example, if the loading pressure needs to be greater than the vacuum pressure that the bulb can generate, such as may be needed to pass the sample through a filter, or if the sample liquid is very viscous, or if the volume of the total sample is greater than what could be easily accommodated via bulb aspiration, or if the nature of sample collection does not accommodate the bulb aspiration method, such as a blood sample that has been collected via a vein puncture.

The syringe loading method has the advantages of increased pressure generation, increased volume delivery, the ability to pass the liquid through a filter, and the potential for more controllable liquid delivery and for multiple liquid delivery, etc. The disadvantages include slightly more complex sample loading and more parts to work with.

The valving methodology would be the same, where air is allowed to vent from the testing kit as it fills with liquid, but the valves 347 prevent the liquid from leaking out of the reaction chambers 342, which would cause imprecise reactions and possibly incorrect filling of all chambers.

The air, venting from the testing kit as it is filled with liquid, would be allowed to escape through channels 348 downstream of the valves that lead to the surface of the device through a venting port 341 and open to the atmosphere.

A syringe could also be used similarly to a bulb where the syringe is connected downstream of the reaction chambers and the aspiration of the syringe could be used to fill the reaction chambers. But in this case, as with the bulb, the aspiration force would only reach atmospheric pressure.

FIG. 14 shows a testing kit 360 with a syringe connection 361 where the syringe is used to aspirate the sample into the testing kit instead of an integrated bulb.

Similar to a syringe, a pipette could be used to aspirate the sample into the testing kit. Some electronic pipettes, such as the EDP1™ Electronic Pipette by Rainin Instruments, have multi-step aspiration functions which would facilitate multiple liquid loading and timed incubation that may be needed in a two-step process.

It is also conceivable that a needle could be fixed onto the tip of the testing kit for drawing a sample from a vein or for puncturing a membrane to extract a sample from a sealed container. FIG. 15 illustrates a single sample single test circuit (singlet) 380 with a needle 384 and a fitting for either a syringe or pipette 381 for sample aspiration. An integrated bulb could also be used.

Multi-Sample Testing Kits: Similar to a multi-tip pipette, a multi-tip multiple sample testing kit is also possible. FIG. 16 shows a 3-tip testing kit 400 where each sample is loaded into a single reaction chamber 405. This drawing shows an integrated aspiration bulb 401 whereas FIG. 17 shows the same circuit design 420 but with a fitting for syringe or pipette aspiration 421. Potentially many tips are possible and their size and location can vary as needed for the application. Tips 9 mm apart, for example, could be useful for analyzing samples located in the wells of a 96-well microtiter plate. Although the drawings show only single reaction chambers for each sample, much more complex systems can be used as well.

Examples of Use: An example of the use of the testing kit platform described according to the present invention is the analysis of a human saliva sample. A sponge may be placed in the mouth of the person whose saliva is to be collected. The sponge may be a simple sponge or one specialized for saliva collection and connected to a stick or handle, such as Saliva Sampler™ from Saliva Diagnostic Systems (SDS) Inc. After several minutes of collection, the sponge is removed from the mouth and placed in a syringe. In some instances the handle connected to the sponge may also be the plunger of the syringe. The syringe is connected to the testing kit, possibly through a bubble filter, and the sample is manually dispensed into the testing kit. In the case of a single step analysis the sample is dispensed into the testing kit until all reaction chambers have been filled.

In the case of a 2-step process, namely reaction and washing steps, the saliva is dispensed into the testing kit until the reaction chambers are filled and then the pressure on the syringe is released for as long as may be needed for the reaction to come to completion. During this saliva loading process as each reaction chamber is filled with the sample, eventually the saliva will engage the valve material connected to the reaction chamber via a channel. The valve material will react with the saliva and prevent, temporarily, further flow of the sample down the channel. The remaining pressure on the syringe and flow of saliva into the testing kit is shunted to fill the remaining reaction chambers. Once all reaction chambers are filled completely, the dispensing of the sample is stopped.

If the excess saliva in the syringe can also be used as a washing buffer, then the pressure on the plunger for dispensing the saliva is resumed and the temporary valves holding the saliva in the reaction chambers are breached until the saliva has filled the dead-volume wells and is stopped by the permanent valves downstream of the dead-volume wells.

If the reaction chamber volume is 20 μL, it may be desirable for the washing volume to be about five times this volume, that is 100 μL. If there are three reaction chambers the total volume of the reaction chambers and dead volume washing wells may be approximately 360 μL. Assuming the total remaining dead volume of the channels in the testing kit is another 100 μL, the total volume of the device may be approximately 460 μL. The aforementioned Saliva Sampler™ from SDS is designed to collect a sample volume of approximately 1 mL, which is adequate for this example.

FIG. 18 illustrates a three reaction chamber two-step device 440 with a syringe fitting 445 on the inlet 446 for sample loading. FIG. 18 shows the use of temporary valves 447 for sample distribution and incubation in the reaction chambers 443, and permanent valves 448 and dead-volume wells 442 for washing out the reaction chambers.

In the case where the washing buffer must be different than the sample, such as for the delivery of a reaction termination reagent, either the saliva syringe will need to be removed and another syringe connected to the same inlet port, or a second inlet port may be provided and the sample syringe can remain attached.

When multiple inlets for multiple syringes are used, care must be taken that air is not introduced into the testing kit which can disrupt fluid flow and valve operation. If a second inlet is provided, the first liquid may be used to prime the channel connecting the second inlet to the main device flow channel leading to the reaction chambers.

This priming may be accomplished by having an air filter cap, such as the Sims Portex Filter-Pro Air Bubble Removal Device™ connected to the second inlet. This is an external valve cap that allows air to flow through, but will seal when liquid enters into it. The cap can then be removed and the second syringe connected, or there could be an external two-way valve connected to the second inlet where at first the valve is open to the air filter cap. Once all air is vented and liquid reaches the cap, the external valve can be switched over to the inlet connecting the second syringe to the testing kit.

When the second liquid is dispensed into the testing kit, it is possible it will simply flow out the first inlet rather than into the reaction chambers. This can be prevented by capping the first inlet once the sample is dispensed into the testing kit, or by placing an isolation valve within the testing kit downstream of the first inlet, but upstream of where the second inlet connects onto the main flow channel.

FIG. 19 shows a testing kit 460 with two syringe attachment points 472 and 468 for sample loading and two-step liquid reagent delivery. The first inlet channel includes a pass-through valve 471 to isolate it from flow coming from the second inlet 468.

As shown in FIG. 20, there may be a case where the sample is loaded via a bulb aspiration process, and then a syringe is connected for a secondary reagent delivery or washing step. This can be done by having the testing kit inlet 486 accommodate a syringe connection 485. In this case, however, the bulb 481 must have a venting port 491 that is uncovered when the syringe is dispensing a liquid into the testing kit.

In each of these cases there is no need to limit the testing kit platform to two inlets or two liquid delivery steps. More inlets and more liquid delivery steps may be added and the flow controlled by careful attention to the volume of liquid dispensed and the use of multiple temporary valves in series with each other and in parallel with other reaction chamber flow paths.

Sample Collection: All previous examples have described sample collection happening independently of the testing kit. For example, blood may be drawn and collected by a syringe and then loaded or delivered to the testing kit through the syringe fitting inlet. Saliva can be collected with a sponge and delivered through a syringe. Or the sample is already collected in some kind of appropriate vessel, such as a glass or beaker, and then the tip of the device is inserted into the liquid and drawn up via bulb aspiration.

It is also possible to mold a collection device into the testing kit, similar to how the bulb is molded into the kit. The collection device could be a small scoop or reservoir connected to the tip of the testing kit. It would be large enough to contain the volume of sample needed for filling the kit, and it would be in fluid communication with the kit via a channel that draws the liquid from the bottom of the reservoir into the kit.

This design could be used to collect urine or saliva and any other liquid that may benefit from an integrated collection capability. For example, FIG. 21 shows a three reaction chamber saliva testing kit 500 where the bulb 507 would be depressed and then the tip of the device containing a sample reservoir 504 held under the tongue for a few seconds to minutes until it is filled. It would be withdrawn to verify the volume is sufficient, and then the bulb is slowly released drawing the saliva into the kit. In the case where the reaction chambers 503 are approximately 20 μL each, a total volume collected of 100 μL should be sufficient and easily achieved. The user could also spit into the reservoir 504 if desired.

What is claimed is:

1. A method for stopping fluid flow within a device, comprising:
causing a liquid to enter a flow path through a device;
the liquid reacting with a material located along the flow path;
the liquid and the material generating a product that impedes further flow of the liquid through the device.

2. The method according to claim 1, wherein the liquid reacting with the material comprises the material swelling in the presence of the liquid.

3. The method according to claim 1, wherein the liquid and the material generating a product comprises the material polymerizing in the presence of the liquid, forming a semi-solid material that impedes fluid flow.

4. The method according to claim 1, wherein the liquid and the material generating a product comprises generating a viscous product that impedes liquid flow.

5. The method according to claim 1, wherein the material is configured to dissolve, thereby reversing effect of impeding fluid flow.

6. The method according to claim 1, wherein the material comprises components that break down the product, thereby reversing the effect of impeding fluid flow.

7. The method according to claim 6, wherein the components that break down the product comprise at least one of: amylases, cellulases or xylanases.

8. A valve for stopping liquid flow, comprising a material configured to allow air to pass through the valve and configured to stop flow of a liquid through the valve upon contact with the liquid.

9. The valve according to claim 8, wherein the material swells upon contact with the liquid to form a liquid flow barrier.

10. The valve according to claim 8, wherein the material polymerizes upon contact with the liquid to form a liquid flow barrier.

11. The valve according to claim 8, wherein the material dissolves in the presence of the liquid, forming a viscous product that impedes liquid flow.

12. The valve according to claim 8, wherein the material comprises acrylamide hydrogel, polyvinyl-chloride, or starch.

13. The valve according to claim 8, wherein the material comprises EnviroBond 403™, or a multi-part adhesive.

14. The valve according to claim 8, wherein the material comprises at least one of: carboxymethyl cellulose, starch, a cross-linking sugar, or Ficoll™ brand sugar.

15. The valve according to claim 8, wherein the material comprises components configured to enhance or accelerate the reaction of other components in the material to expedite impedance of liquid flow.

16. An apparatus, comprising:
a reaction chamber;
an inlet for receiving a fluid;
a fluid channel leading from the inlet to the reaction chamber;
a valve connected to the reaction chamber, the valve comprising a material configured to allow air to pass through the valve and configured to stop flow of the fluid through the valve upon contact with the fluid;
an outlet; and
an exit channel connecting the outlet with the valve.

17. The apparatus according to claim 16, wherein the material comprises at least one of: acrylamide hydrogel, polyvinyl-chloride, starch, EnviroBond 403™, a multi-part adhesive, carboxymethyl cellulose, starch, a cross-linking sugar, or Ficoll™ brand sugar.

18. The apparatus according to claim 16, wherein a section of a normal flow path of the fluid containing the material comprises a material chamber that contains the material.

19. The apparatus according to claim 18, wherein the material chamber further comprises features that prevent the material from leaving the material chamber.

20. The apparatus according to claim 19, wherein the features comprise narrow inlet channels entering and narrow outlet channels leaving the material chamber.

21. The apparatus according to claim 19, wherein the features comprise ridges configured to contain the material within the material chamber.

22. The apparatus according to claim 16, wherein the reaction chamber comprises reagents for performing a chemical or biological reaction with the incoming fluid.

23. The apparatus according to claim 16, wherein the reaction chamber is configured to be of specific dimensions in order to contain a specific volume of the incoming fluid to be analyzed.

24. The apparatus according to claim 16, further comprising a plurality of fluid channels each leading from the inlet to separate reaction chambers each reaction chamber connected to a separate valve along a separate exit channel to the outlet, each separate reaction chamber containing reagents for performing different chemical or biological reactions on the incoming fluid.

25. The apparatus according to claim 24, wherein each of the different chemical or biological reactions are configured to elucidate different biochemical information about the incoming fluid.

26. The apparatus according to claim 24, wherein some of the different chemical or biological reactions are configured to act as positive or negative controls for the other different chemical or biological reactions within the apparatus.

27. The apparatus according to claim 24, wherein some of the different chemical or biological reactions are configured as calibration standards for other different chemical or biological reactions within the apparatus.

28. The apparatus according to claim 24, wherein some of the different chemical or biological reactions are configured to identify the presence of a compound in the incoming fluid that may interfere with other different chemical or biological reactions.

29. The apparatus according to claim 22, wherein the reagents in some of the reaction chambers are of the same composition as in the other reaction chambers, but of different masses in order to elucidate the concentration of an analyte in the incoming fluid such as in a titration reaction.

30. The apparatus according to claim 22, wherein the reagents comprises at least one of: compressed powders, lyophilized pellets, coated beads, enzymes, test strips, fluorescent materials or dyes.

31. The apparatus according to claim 22, wherein the reagents are at least one of:
immobilized on a surface of the reaction chamber or the fluid channel; or
free standing within the reaction chamber; or
encapsulated in a soluble coating.

32. The apparatus according to claim 22, wherein the reagents are either in standard form or chemically modified to be soluble in the incoming fluid.

33. The apparatus according to claim 22, wherein the reagents are physically modified prior to placement in the reaction chamber.

34. The apparatus according to claim 24, wherein the dimensions of each of the separate reaction chambers vary for each reaction chamber, allowing for different reactions with different volumes of the incoming fluid.

35. The apparatus according to claim 16, wherein the inlet further comprises a fitting for a syringe or pipette for dispensing a sample fluid into the apparatus using a syringe or pipette.

36. The apparatus according to claim 16, wherein the outlet further comprises a fitting for a syringe or pipette for drawing a vacuum at the outlet.

37. The apparatus according to claim 16, further comprising a fitting for a bubble or particle filter on the inlet.

38. The apparatus according to claim 16, further comprising an aspiration bulb connected to the outlet and configured to draw a vacuum at the inlet until the valve is closed.

39. The apparatus according to claim 38, wherein the aspiration bulb further includes a hole through the aspiration bulb for relieving vacuum pressure within the aspiration bulb.

40. The apparatus according to claim 16, further comprising an integrated liquid sample collection device.

41. The apparatus according to claim 16, comprising two parts that are sealed together to form the apparatus.

42. The apparatus according to claim 41, wherein the two parts are sealed together by at least one of: ultrasonic welding, solvent bonding, or lamination using transfer film, double sided tape or screen printed adhesive.

43. The apparatus according to claim 16, further comprising electrical elements for fluid manipulation, or sensing.

44. The apparatus according to claim 43, wherein sensing comprises sensing at least one of: liquid component, reaction product, chemical parameter or physical parameter.

45. The apparatus according to claim 16, further comprising optical elements for physical or chemical parameter detection.

46. The apparatus according to claim 45, wherein the electrical elements are configured to interface with an external instrument for external control of an electrical element function.

47. The apparatus according to claim 41, wherein at least one of the two parts comprise plastic.

48. The apparatus according to claim 16, wherein the material is configured to completely impede fluid flow for a specific amount of time, after which it allows liquid to flow through the valve.

49. The apparatus according to claim 16, wherein the material is configured to allow a specific volume of fluid to flow past it before it responds to or reacts with the fluid sufficiently enough to permanently or temporarily, partially or completely, impede further fluid flow through the apparatus.

50. The apparatus according to claim 16, wherein the material is configured to chemically, or thermally, or physically isolate a volume of the fluid downstream from the valve from the fluid, upstream from the valve.

51. The apparatus according to claim 16, further configured to allow an aspiration force applied to the outlet to draw fluid or air into the apparatus through an alternative input in the apparatus connected to the liquid flow path downstream from the valve.

52. The apparatus according to claim 16, wherein the inlet is connected to a needle or a fitting for a needle configured for puncturing a membrane to gain access to a fluid.

53. The apparatus according to claim 16, wherein the fluid comprises at least one of: fresh water, salt water, drinking water, stored water, waste waster, aquarium water, aquaculture water, ship ballast water, saliva, urine, blood, blood products, liquid food, liquefied food, alcoholic beverage, non-alcoholic beverage, industrial chemicals, solvents, fuels, petroleum products, or oils.

54. A process for performing a chemical or biological reaction on a liquid sample, comprising:
providing a liquid analysis device, comprising:
a reaction chamber containing a reagent;
an inlet for receiving a liquid;
a liquid channel leading from the inlet to the reaction chamber;
a valve connected to the reaction chamber, the valve comprising a material configured to allow air to pass through the valve and configured to stop flow of the liquid through the valve upon contact with the liquid;
an aspiration bulb; and
an exit channel connecting the aspiration bulb with the valve, the aspiration bulb configured to draw a vacuum at the inlet until the valve is closed;
depressing the aspiration bulb;
placing the inlet of the device into a liquid sample; and
releasing the aspiration bulb, thereby aspirating the liquid into the liquid analysis device where reagents stored within the reaction chamber react with the liquid.

55. The process according to claim 54, wherein placing is performed either before or after depressing.

56. A process for depositing a reactive or responsive material into a liquid analysis, storage or processing device, the process comprising:
making a slurry of the material in a volatile liquid that the material does not respond or react with to any great degree;
depositing the slurry into a selected region of the device meant to contain the material;
allowing the volatile liquid to evaporate leaving the reactive or responsive material on the selected region of the device.

57. A device for performing a panel of chemical or biological analysis, comprising a single inlet and a single aspiration or dispensing source, wherein the single inlet branches into multiple flow channels and wherein each flow channel includes a section that contains one or more materials that respond to or react with the liquid to generate a product that, either permanently or temporarily, partially or completely impedes the further flow of the liquid through the flow channel.

58. The apparatus according to claim 48, wherein the valve configured to completely impede fluid flow for a specific amount of time, is followed by and in fluid communication with a second valve that is configured to stop liquid flow, the second valve comprising a material configured to allow air to pass through the valve and configured to stop flow of a liquid through the valve upon contact with the liquid.

59. The apparatus according to claim 58, wherein the placement of the valve and second valve are designed to facilitate a secondary process within the apparatus.

60. The apparatus according to claim 59, wherein the secondary process includes a secondary reagent or liquid delivery into the apparatus, or a washing of a reaction chamber within the apparatus.
